(19) United States
(12) Patent Application Publication
(10) Pub. No.: US 2023/0390046 A1
(43) Pub. Date: Dec. 7, 2023

(54) KIDNEY STONE MANAGEMENT

(71) Applicant: GYRUS ACMI, INC. D/B/A OLYMPUS SURGICAL TECHNOLOGIES AMERICA, Westborough, MA (US)

(72) Inventors: Thomas J. Holman, Princeton, MN (US); Nikhil M. Murdeshwar, Maple Grove, MN (US)

(21) Appl. No.: 18/032,752

(22) PCT Filed: Oct. 20, 2021
(86) PCT No.: PCT/US2021/071950

§ 371 Date: Apr. 19, 2023

Related U.S. Application Data
(60) Provisional application No. 63/094,051, filed on Oct. 20, 2020.

(51) Int. Cl. A61F 2/04; A61F 2/00; A61B 5/20

(52) U.S. Cl. CPC A61F 2/04; A61F 2/0095; A61B 5/201; A61F 2002/048; A61F 2220/0016; A61F 2210/0057

(57) ABSTRACT

Devices, systems, and methods for kidney stone management are provided. A device can include a medical apparatus comprising a kidney stone blocker configured to (i) remain in a human kidney after deployment and (ii) prevent passage of a kidney stone greater than a predetermined size into a ureter while allowing urine to pass to the ureter, and an elongated, tubular member mechanically coupled to the kidney stone blocker and configured to be situated in the ureter.

### RELATED APPLICATION

This application claims the benefit of priority to U.S. Provisional Patent Application No. 63/094,051 titled “Kidney Stone Management” and filed on Oct. 20, 2020, the entire contents of which is hereby incorporated herein by reference in its entirety.

### TECHNICAL FIELD

Embodiments described herein generally relate to medical devices, systems, and techniques for kidney stone management that help in preventing pain experienced from a kidney stone episode.

### BACKGROUND

People who have had kidney stones are susceptible to recurrence. The recurrence rate of secondary kidney stone formation ranges from approximately about 10% to about 75%. The likelihood of secondary kidney stone formation is affected by the number of previous stone episodes and the time since the last kidney stone episode.

A kidney stone episode can be experienced at any time. A kidney stone episode can include a significant amount of pain, to the point of debilitation. The kidney stone episode can thus significantly disrupt a daily life. People who have experienced a kidney stone episode often worry about the next kidney stone event occurring. These people can be troubled by anticipating the accompanying pain, if and when the next kidney stone episode arises.

### SUMMARY

Embodiments help prevent kidney stones of a minimum size from entering a ureter of a patient. Embodiments can indicate when a stone is present. Such an indication can help a user schedule when to get treatment for the kidney stone. Embodiments thus help prevent pain caused from kidney stones and provide kidney stone management.

A medical apparatus can include a kidney stone blocker configured to (i) remain in a human kidney after deployment and (ii) prevent passage of a kidney stone greater than a predetermined size into a ureter while allowing urine to pass to the ureter. An elongated, tubular member can be mechanically coupled to the kidney stone blocker and configured to be situated in the ureter. The kidney stone blocker can include one or more mesh screens. In an embodiment in which the kidney stone blocker includes multiple mesh screens, at least two of the mesh screens can be configured to block kidney stones of respective, different minimum sizes. The multiple mesh screens can be arranged such that a first mesh screen of the multiple mesh screens is situated further from the ureter than a second mesh screen of the multiple mesh screens, the second mesh screen configured to prevent passage of smaller kidney stones than the first mesh screen.

An anchor can be mechanically coupled to the member. The anchor can be configured to be retained in a bladder. The member can be longitudinally extendable or elastic. The kidney stone blocker can include at least one of a barb, hook, tine, or stem configured to engage with the kidney and retain the kidney stone blocker in the kidney. A stone breaking device can be situated in the member. The stone breaking device can be configured to break the kidney stone into a plurality of smaller kidney stones. The stone breaking device can include an optical device, an audio device, or a mechanical device.

The kidney stone blocker can be expandable so as to fit through the ureter in a non-expanded form and to be retained in the kidney in an expanded form. The kidney stone blocker includes a hydrophilic coating.

A method can include providing a stone blocker into a ureter. The stone blocker can be formed with at least one opening for preventing passage of a kidney stone larger than the openings into the ureter while allowing urine to pass to the ureter. The method can include positioning the stone blocker within a kidney. The method can include maintaining the stone blocker within the kidney to retain, in the kidney, the kidney stone larger than the at least one opening. The method can further include expanding, by activating an expansion mechanism, the stone blocker after positioning the stone blocker within the kidney. The expansion mechanism includes at least one of a balloon or a spring-loaded wire.

The method can further include detecting whether the kidney stone is present, and responsive to detecting the kidney stone is present, breaking up the kidney stone into smaller kidney stones. The method can further include detecting whether the kidney stone is present. The method can further include responsive to detecting the kidney stone is present, transmitting an indication to a communication device that the kidney stone is present.

A system can include a kidney stone detection device configured to determine whether a kidney stone is present in a kidney of a patient and a kidney stone blocker configured to (i) remain in a kidney after deployment and (ii) prevent passage of the kidney stone into a ureter while allowing urine to pass to the ureter. The kidney stone detection device can produce sound waves or light to determine whether a kidney stone is present in a kidney of a patient. The system can further include an elongated, tubular member mechanically coupled to the kidney stone blocker, the member sized to be situated in a ureter. The kidney stone blocker can be expandable so as to fit through the ureter in a non-expanded form and to be retained in the kidney in an expanded form.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 illustrates, by way of example, a diagram of an embodiment of an implanted kidney stone blocker.

FIG. 2 illustrates, by way of example, a diagram of an embodiment of the kidney stone blocker that is anchored in the kidney.

FIG. 3 illustrates, by way of example, a diagram of an embodiment of a portion of a kidney stone blocker.

FIG. 4 illustrates, by way of example, a diagram of an embodiment of a portion of another kidney stone blocker.

FIGS. 5, 6, and 7 illustrate, by way of example, respective stages of a kidney stone blocker implant process.

FIG. 8 illustrates, by way of example, a diagram of an embodiment of a system for kidney stone blocking and detection.

FIG. 9 illustrates, by way of example, a block diagram of an embodiment of a system for kidney stone reduction using a kidney stone breaker.

FIG. 10 illustrates, by way of example, a block diagram of an embodiment of the system of FIG. 9 after the kidney stone breaking device has broken the kidney stone into smaller pieces.

FIG. 11 illustrates, by way of example, a block diagram of an embodiment of a stone detecting system.

FIG. 12 illustrates, by way of example, a block diagram of an embodiment of a machine (e.g., a computer system) to implement one or more embodiments.

### DESCRIPTION OF EMBODIMENTS

A device, system, or method for kidney stone management can predict and manage a kidney stone event. The kidney stone management can help eliminate a kidney stone event for patient. The kidney stone management can allow a patient to handle the kidney stone event on their own time. The kidney stone management can allow the patient to handle the kidney stone event without experiencing the pain associated with the kidney stone travelling in the ureter towards the bladder.

The kidney stone management can include a stone blocker or a barrier that allows urine from the kidney to pass, while creating a barrier to kidney stones. The barrier can help prevent the kidney stone from reaching the ureter. The barrier can help enable scheduled analysis of stone development at a time when the stones are painlessly retained within the kidney.

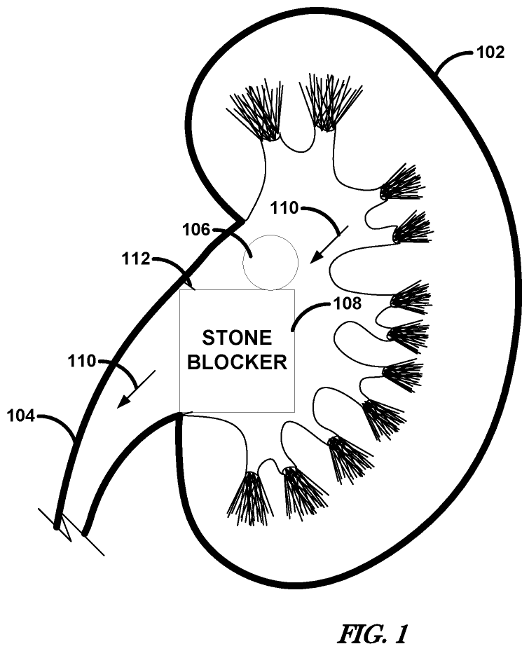

The following disclosure can be used with a number of different types of kidney stone management devices, systems, or techniques. One example for illustration shown in FIG. 1 is a stone blocker situated in a kidney.

A component with a reference number with a suffix represents a specific instance of a component with the same reference number without the suffix. For example, a stone blocker 108A is a specific instance of a stone blocker 108.

FIG. 1 illustrates, by way of example, a diagram of an embodiment of an implanted kidney stone blocker 108. The kidney stone blocker 108 is situated, at least partially, in a kidney 102. The kidney stone blocker 108 can be situated, at least partially in the ureter 104. The kidney stone blocker 108 is sized and shaped to block a kidney stone 106 from entering the ureter 104. The kidney stone blocker 108 is configured to retain the kidney stone 106 in the kidney 102. In some embodiments, the stone blocker 108 can be configured to retain any kidney stone above a threshold size in the kidney 102 and let any kidney stone below the threshold size through to the ureter 104.

The kidney stone blocker 108 is liquid permeable, such as to allow filtered urine to flow through to the ureter 104 (indicated by arrows 110). The permeability of the kidney stone blocker 108 can be provided by a mesh structure of the kidney stone blocker 108, holes in the kidney stone blocker 108, or other gaps through which fluid from the kidney 102 can flow. The stone blocker 108 can be formed using nitinol (or other shape memory material), a polymer (e.g., a polymer with holes (e.g., laser cut holes) that allow urine to pass therethrough), braided filament (e.g., 16, 32, or 64 braided filament), or other deformable, biocompatible material. The kidney stone blocker 108 can include a hydrophilic coating. Examples of biocompatible hydrophilic coatings include polyvinylpyrrolidone (PVP), polyurethane, peracetic acid (PAA), polyethylene oxide (PEO), polysaccharide, or the like. The hydrophilic coating can absorb fluid and cause the stone blocker 108 to expand. The hydrophilic coating can be reactive to a fluid in the urine. The stone blocker 108 can expand into the renal pelvis, by way of the hydrophilic coating. The stone blocker 108 can include an expanding foam (e.g., an open cell material) that expands, retains stones in the kidney 102, and also lets urine therethrough.

The kidney stone blocker 108 can include an attraction mechanism for retaining the stone 106. The attraction mechanism can help prevent the stone 106 from moving and reduce the chances the stone 106 migrates into the ureter 104. At attraction mechanism can include an electrostatic attractor, a chemical attractor, or the like.

The kidney stone blocker 108 can be shaped to retain the stone 106. As urine from the kidney 102 is pushing the stone 106 towards the ureter 104, a dimple, indent, depression, or other pre-formed shape in the kidney stone blocker 108 can help retain the stone 106. The kidney stone blocker 108 can include, for example, a cone, pyramid, or other shape that tapers towards the ureter 104, such as to catch and retain the stone 106. The kidney stone blocker 108 can include a malleable fabric that deforms under the force of the stone 106. The deforming of the fabric can help retain the stone 106 in the kidney 102 and help prevent the stone 106 from travelling to the ureter 104.

The stone blocker 108 can include a retention mechanism 112 extending therefrom. The retention mechanism 112 can include a tine, barb, stem, or other protrusion configured to engage with the kidney 102 and retain the stone blocker 108 in the kidney 102. Additionally, or alternatively, a surface of the stone blocker 108 that interfaces with or otherwise contacts the kidney 102 can be rough, jagged, or otherwise non-smooth. The non-smooth surface can include a directional or non-directional slip resistant surface that helps retain the stone blocker 108 in the kidney 102. The retention mechanism 112 can help prevent movement of the stone blocker 108. The retention mechanism 112 can help prevent the kidney stone 106 from reaching the ureter 104. This is, at least in part, because the retention mechanism 112 can help retain the stone blocker 108 in its location. The retention mechanism 112 can also help keep the stone blocker 108 in a blocking orientation. The retention mechanism 112 can help retain the sides of the stone blocker 108 in a position that prevents the kidney stone 106 from getting into the ureter 104.

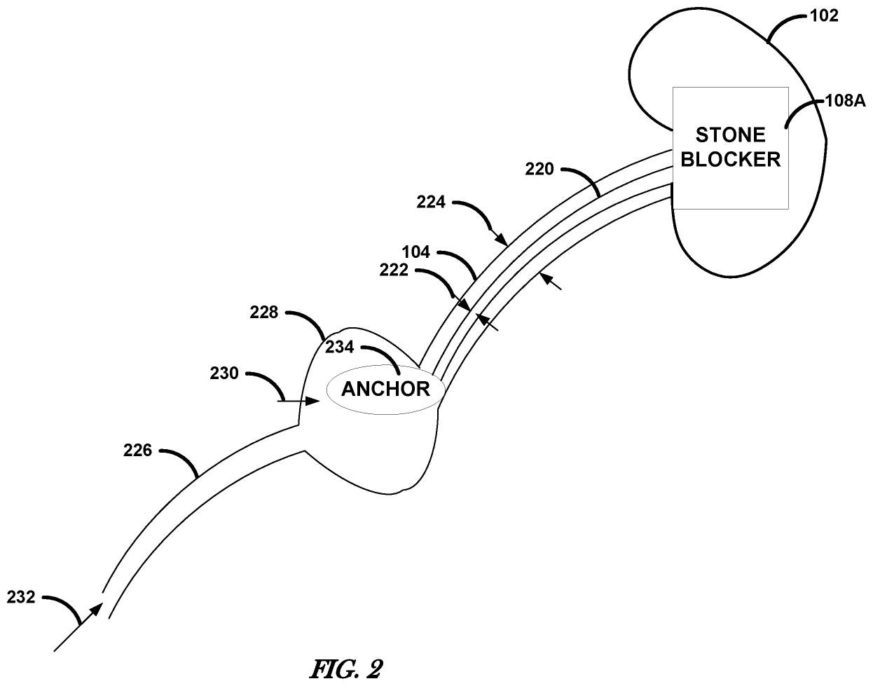

FIG. 2 illustrates, by way of example, a diagram of an embodiment of a kidney stone blocker 108A that is anchored in the kidney 102. The kidney stone blocker 108A can include an elongated member 220 attached thereto. The elongated member 220 can be solid or hollow. The elongated member 220 can include an outer diameter 222 that is smaller than an inner diameter (indicated by arrows 224) of the ureter 104.

The elongated member 220 can be inserted with the kidney stone blocker 108 through a urethra 226 (indicated by arrow 232) or an opening in a bladder 228 (indicated by arrow 230). An anchor 234, sized and shaped to be situated in the bladder 228, can help retain the kidney stone blocker 108A in place. The anchor 234 can be mechanically coupled to an end of the elongated member 220 that is opposite the kidney stone blocker 108.

The elongated member 220 can include a ridge, bump, barb, stem, rough surface, or other configuration for securing the elongated member 220 in the ureter 104. The elongated member 220 can extend from the stone blocker 108 to the anchor 234. The elongated member 220 can be longitudinally extendable or contractible, such as by forming the elongated member 220 using an elastic, or otherwise longitudinally deformable material.

The anchor 234 (and the direction of fluid flow from the kidney 102 indicated by arrow 110 in FIG. 1) can help prevent the kidney stone blocker 108 from wandering further into the kidney 102. The size and shape of the kidney stone blocker 108 can help prevent the kidney stone blocker 108 from wandering into the ureter 104.

The anchor 234 can include a mechanism for expanding, contracting, or both. For example, the anchor 234 can have a balloon-like inflating or deflating action. In another example, the anchor 234 can include a suture attached to a retaining structure that, when released, causes the anchor 234 to expand. Another suture, or the like, can be pulled or otherwise altered to cause the anchor 234 to contract.

The member 220 can help facilitate delivery of the stone blocker 108A. The stone blocker 108A can be situated in the member 220 in a compressed or non-expanded configuration. The stone blocker 108A can conform to an inner wall of the member 220 while inside the member 220. The stone blocker 108A can be pushed through the member 220. As the stone blocker 108A exits the member 220, or after the stone blocker 108A exits the member, it can expand to a desired shape. The desired shape can be achieved using a spring, shape memory material, wire, or the like.

In some embodiments, the stone blocker 108A can be delivered independent of the member 220. The stone blocker 108A can be collapsible or deformable, so as to fit through the ureter 104 and be delivered to the kidney 102. The stone blocker 108A can include a wire, filament, or the like, attached thereto that can be pulled to retrieve the stone blocker 108A from the kidney 102.

The stone blocker 108A can have a plurality of finger-like members extending radially from a nucleus. The finger-like members can be collapsible or deformable, similar to a “koosh ball”. The kidney stone 106 can get tangled in the finger-like members. Then, as the kidney stone blocker 108A is retrieved, the kidney stone 106 can be pushed against finger-like members. The finger-like member can break up the kidney stone 106 into smaller pieces that are also tangled in the finger-like members. The smaller pieces can then be passed through urination or retrieved through removal of the kidney stone blocker 108A. The finger-like members can extend to fill a space in which they are situated. The finger-like members can collapse when a force directed towards the center of the stone blocker 108A is asserted on the finger-like members. This allows a stone blocker 108A with the finger-like member to fit through a smaller volume, such as the ureter 104, and expand to fill more of a volume when situated in a larger volume, such as the kidney 102.

FIG. 3 illustrates, by way of example, a diagram of an embodiment of a portion of a kidney stone blocker 108B. The kidney stone blocker 108B includes a structure 330 with gaps or other openings 336 to allow urine to flow through. The structure 330 includes openings 336 with a dimension 332 sized and shaped to prevent the kidney stone 106 traveling through to the ureter 104. Preventing the kidney stone 106 flowing through can include making the dimension 332 (size of the opening 336) less than a corresponding dimension 334 of the kidney stone 106. The kidney stone 106 can be one with a size that is one, two, three, or more or fewer standard deviations below an average size kidney stone, or the like.

While kidney stone blocker 108B and the openings 336 therein are illustrated as generally rectangular, the kidney stone blocker 108B or openings 336 can include a different shape and can be shaped different from each other. Example shapes includes other polygons, irregular shapes, ellipses, a three-dimensional shape, or the like. The shape of the stone blocker 108B, in a deployed state, can include a perimeter that engages with a cross-section of the kidney 102 upstream from the ureter 104. The shape of the stone blocker 108B in a contracted state (sometimes called an implantable state or the state in which the stone blocker 108 is implanted) can be generally cylindrical or tubular so as to be insertable in the kidney 102, through the ureter 104.

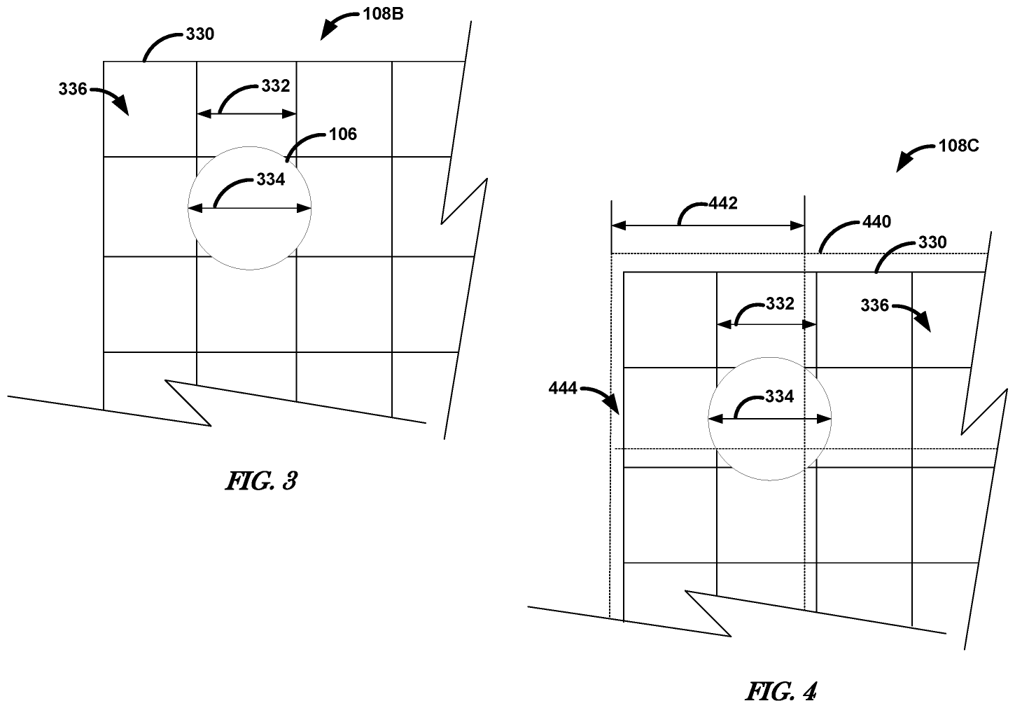

FIG. 4 illustrates, by way of example, a diagram of an embodiment of a portion of another kidney stone blocker 108C. The kidney stone blocker 108C includes the structure 330 with gaps or other openings 336 to allow urine to flow through and block stones of a first size range, with another structure 440 with gaps or other openings 444 to block stones of a different size range than the structure 330. The structure 440 can allow smaller stones to pass while blocking passage of larger stones. A dimension 442 of the structure 440 can be larger or smaller than a corresponding dimension 332 of the structure 330, such as to block stones of a different size. The structures 330, 440 can be mechanically coupled to each other, such as to form a multi-layered stone blocker 108C. While the stone blocker 108C includes two structure layers, more layers, with varying dimensions, can be used in a stone blocker 108. The stone blocker 108C can include a concave or convex structure that accumulates stones in a central or about an edge thereof.

FIGS. 5, 6, and 7 illustrate, by way of example, respective stages of a kidney stone blocker 108 implant process. FIG. 5 illustrates, by way of example, the stone blocker 108 in a contracted state. The contracted state of the stone blocker 108 can allow the stone blocker 108 to move through the ureter 104. In FIG. 5 the stone blocker 108 is being inserted in the kidney 102 through the ureter 104 (indicated by arrow 554). The kidney stone blocker 108 can be situated around or include an expansion mechanism 550.

The expansion mechanism 550 can include a balloon, wires that are curled, bent, or otherwise force-loaded such that cutting or pulling on a suture or other retention mechanism attached to the wires causes the wires to deploy and expand the stone blocker 108, or the like. The expansion mechanism 550 of FIG. 5 is in the contracted position. In contracted position, the expansion mechanism 550 is able to travel through the ureter 104.

FIG. 6 illustrates, by way of example, a diagram showing the stone blocker 108 after it is situated in the kidney 102. FIG. 6 further illustrates activation of the expansion mechanism 550 and the corresponding expansion of the stone blocker 108. The expansion mechanism 550 can be activated, such as through an activation mechanism 552 attached to the expansion mechanism 550. The activation mechanism 552 can include a conduit through which a fluid (e.g., a liquid or gas) is supplied under pressure to cause the expansion mechanism 550 to enlarge. The activation mechanism 552 can include a suture or the like that can be pulled, cut or otherwise manipulated to cause release of a force-loaded wire, spring, or the like that causes the expansion mechanism 550 to enlarge. In the enlarged state, the expansion mechanism 550 can be retained in the kidney 102 as it can be too large to fit in the ureter 104.

While the description so far regards an expansion mechanism 550 that is separate from the stone blocker 108, the stone blocker 108 can, in some configurations, expand without the expansion mechanism 550. For example, the stone blocker 108 can include a malleable or otherwise deformable structure that can expand, such as by a spring, force-loaded wire, or the like, that conforms to a structure in which it resides.

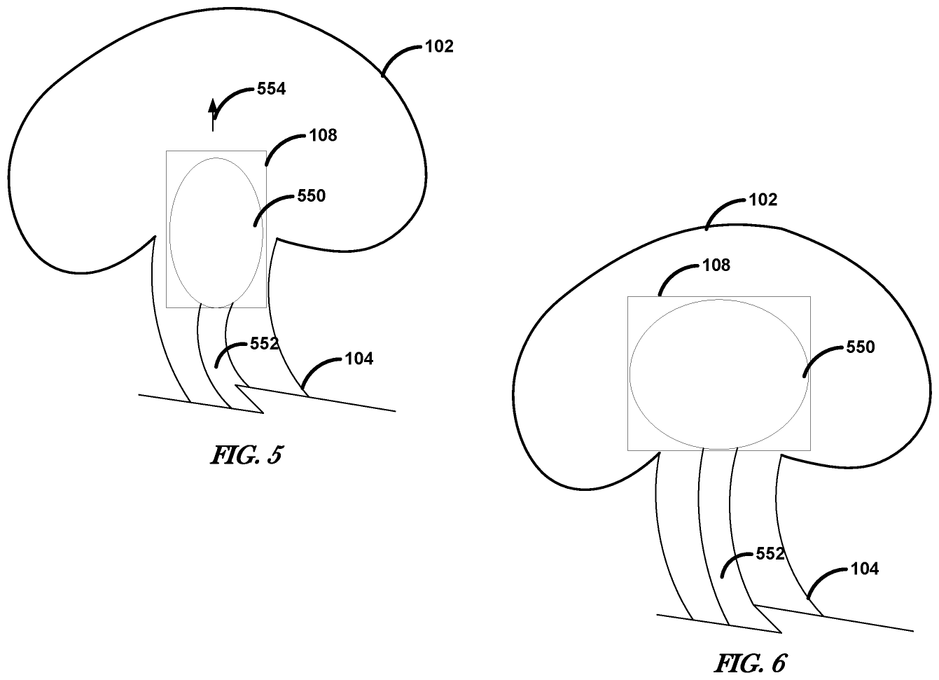

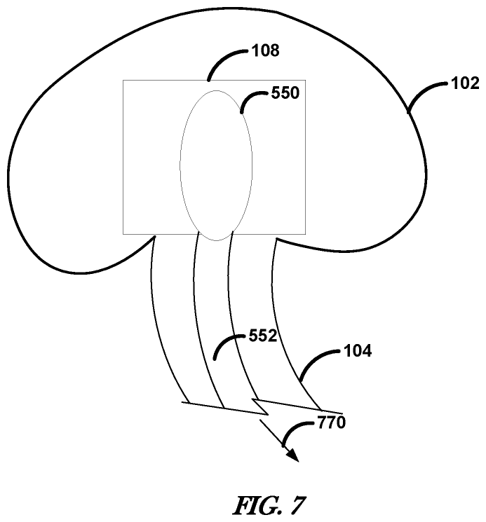

FIG. 7 illustrates, by way of example, a diagram showing the expansion mechanism 550 after it is deflated. Note that deflation is not needed in all cases, such as if the expansion mechanism 550 can be pulled through the ureter 104 in an expanded state. In the deflated state, the expansion mechanism 550 can be pulled through the ureter 104 (indicated by arrow 770), leaving the stone blocker 108 in the kidney 102 (as illustrated in FIG. 1).

The stone blocker 108, may be structured as a medical shunt to facilitate normal flow of fluids from the kidney 102 while keeping stones 106 from reaching the ureter 104. With the stone blocker 108, a patient is spared from the intense pain that can otherwise occur if the stones 106 were allowed to exit the kidney 102 into the ureter 104. The examples depicted include a loop or mesh item as the catcher, but a number of configurations can work to catch a stone 106 while still allowing fluid from the kidney 102 to pass.

The stone blocker 108 can be deployed in any patient at risk of forming kidney stones 106. The stone blocker 108 can be suitable for patients who are at a higher-risk for kidney stones 106. The stone blocker 108 can be implanted during or after a stone removal or fragmentation procedure. Due to high recurrence rates, and the potential for other stones 106 or stone fragments to lurk in the kidney 102, the physician can situate the stone blocker 108 in the patient after removing the stone 106 or a fragmentation procedure.

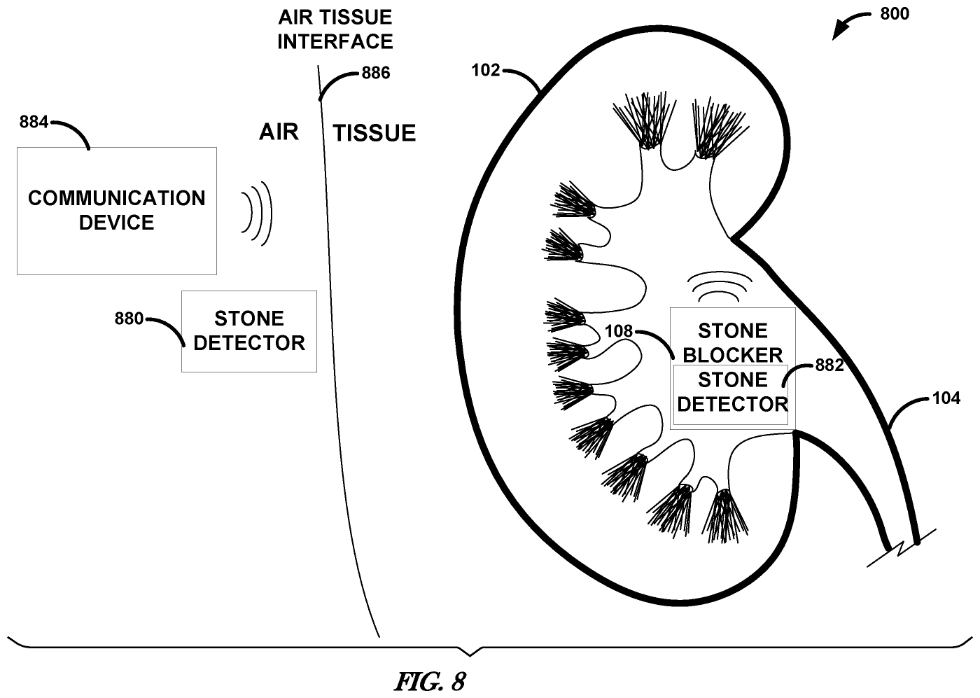

FIG. 8 illustrates, by way of example, a diagram of an embodiment of a system 800 for kidney stone blocking and detection. The system 800 as illustrated includes a stone blocker 108 implanted, at least partially, in a kidney 102. The stone blocker 108 can be implanted to catch or otherwise block a kidney stone 106 from travelling to the ureter 104 while allowing urine or other fluid to flow from the kidney 102 to the ureter 104.

The system 800 can include an internal stone detector 882 or an external stone detector 880. The internal stone detector 882 is configured to detect the stone 106 from within the patient. The external stone detector 880 is configured to detect the stone 106 from outside the patient. Put another way, the internal stone detector 882 detects stones on a tissue side of an air-tissue interface 886 and the external stone detector detects stones from an air side of the air-tissue interface 886.

The stone detector 882 can include a capacitance sensor, an accelerometer, a resistivity sensor, a chemical sensor, other transducer or any suitable sensor capable of detecting the presence of the stone 106 on or around the stone blocker 108. The chemical sensor can include a blood sensor (e.g., iron sensor), or other chemical indicator of the presence of the kidney stone 106. The stone detector 882 can be interrogated, such as by a communication device 884, to provide data indicating whether a stone 106 is detected on or around the stone blocker 108. The stone detector 882 can include communications circuitry configured to provide a response to the interrogation. The stone detector 880 can include a camera, such as can be used during intravenous pyelography (IVP), or other like. Such a stone detector 880 can provide a real-time image to medical personnel to identify whether a stone 106 is in or around the stone blocker 108.

The external stone detector 880 can include an imaging device, such as an ultrasound, a chromatography (CT) scanner, or the like. The external stone detector 880 can include a test strip that detects an amount of iron in the urine, which is indicative of whether there is blood in the urine). The test strip can change color if iron levels indicative of blood presence is detected.

The stone detector 880, 882 can allow a patient to determine whether they have a stone 106 to be removed or not, before going into a procedure for stone removal. The stone detector 880 can include a fluoroscope, an imaging device that can provide a view of the stone 106 after dye ingestion, or the like. The communication device 884 can include any device capable of receiving information from the stone detector 880, 882 and communicating the information to another entity. The communication device 884 can include a display capable of communicating whether the stone 106 is in or around the stone blocker 108. The communication device 884 can include a mobile phone, computer, or a specialized communication device, etc.). In one embodiment, the communication device 884 can send a wireless communication to a medical personnel office (e.g., via email, text, or the like).

Sensing a stone 106 retained by the stone blocker 108 can avoid a need for scheduled appointments to check for stone collection in the kidney 102. With the stone blocker 108 properly deployed, the patient will not experience pain if a renal stone develops. However, the stone detector 880, 882 can recognize the presence of a stone 106. The communication device 884 can notify another device associated with a physician of the apparent need to more directly check for retained stones 106.

One manner of implementing sensing is to enlist the help of the patient to use a product capable of identifying the presence of a captured stone 106. For example, a patient could periodically use the stone detector 880 in the form of a hand-held ultrasound device. The ultrasound device can produce an image that can be manually or automatically sent to medical for analysis, such as by the communication device 884. In another example, a patient can attach or wear the stone detector 880, such as on a belt or other attaching means to position the stone detector 880 at an appropriate location for sensing.

The patient can schedule an appointment sometime after having a stone removal procedure, after having a stone blocker 108 implanted, or after sensing the stone 106. The physician can then check the status of further stone emergence in a scheduled, controlled environment before the patient has to experience pain from another stone 106 event.

FIG. 9 illustrates, by way of example, a block diagram of an embodiment of a system for kidney stone reduction. The system includes a kidney stone breaking device 990 and the stone blocker 108. The kidney stone breaking device 990 can be inserted through the ureter 104. The kidney stone breaking device 990 can be inserted through the member 220 (see FIG. 2). The kidney stone breaking device 990 can be deployed responsive to receiving an indication (at the communication device 884 (see FIG. 8) or from the stone detector 880, 882 (see FIG. 8)) that the kidney stone 106 is present. The kidney stone breaking device 990 can be deployed concurrent with the stone blocker 108 or after deployment of the stone blocker 108. The kidney stone breaking device 990 can be mechanically coupled to the stone blocker 108, such as by a button, loop, hook, wire, or other fastener.

The kidney stone breaking device 990 can include an optical source, a mechanical breaker, or an audio source. The optical source can include a fiber optic cable coupled to a laser, or other light generating mechanism, that produces light that, when incident on the kidney stone 106, breaks the kidney stone 106 into smaller pieces. The mechanical breaker can include a retractable cutting device, such as a forceps, scissors, pliers, or the like. The audio source can include a speaker that generates sound at a frequency that breaks the kidney stone into smaller pieces.

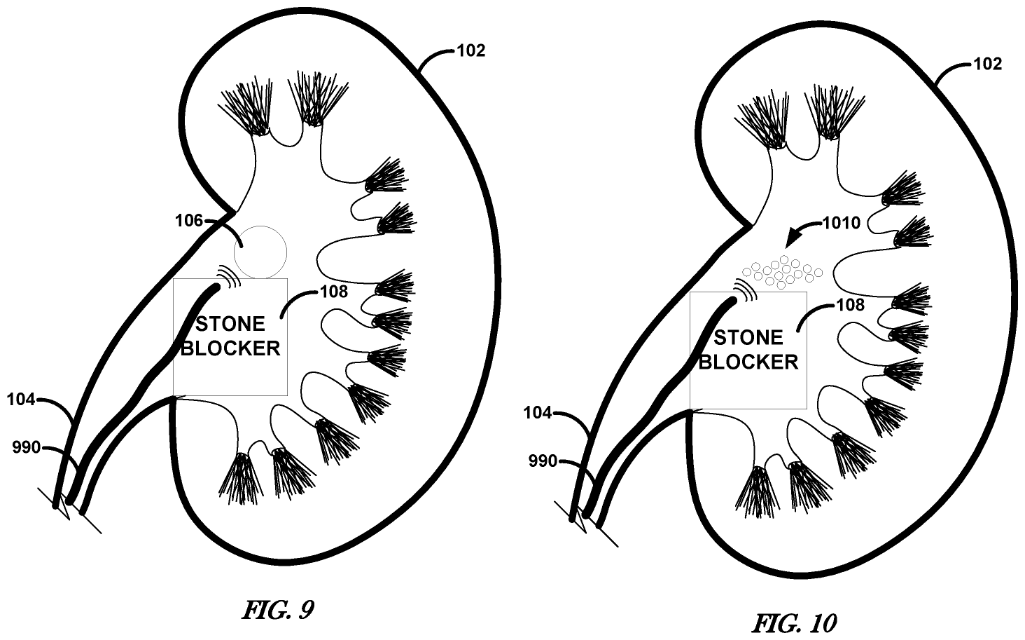

FIG. 10 illustrates, by way of example, a block diagram of an embodiment of the system of FIG. 9 after the kidney stone breaking device 990 has broken the kidney stone into smaller pieces 1010. The smaller pieces 1010 can be smaller than a diameter of the ureter 104 (arrows 224 of FIG. 2 indicate this dimension). One or more of the smaller pieces 1010 can be allowed to pass to the ureter 104 by the stone blocker 108. Additionally, or alternatively, the smaller pieces 1010 can be collected by the stone blocker 108 and removed concurrent with the stone blocker 108. For example, if the stone blocker 108 includes a mesh with openings, the pieces 1010 can be pushed into the stone blocker 108, such as by urine flow toward the ureter 104. The mesh can bend and trap or snare the pieces 1010, allowing the pieces 1010 to be removed concurrent with removal of the stone blocker 108.

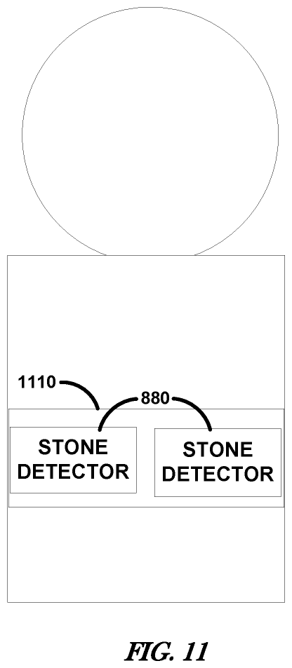

FIG. 11 illustrates, by way of example, a block diagram of an embodiment of a stone detecting system. The stone detecting system includes one or more stone detectors 880 situated in a garment 1110. The garment 1110 can include a shirt, belt, suspenders, band, or the like. The garment 1110 can include one or pockets configured to retain a respective stone detector 880 proximate the kidney of the patient. The patient can continuously or intermittently wear the garment 1110 and operate the stone detector 880. The stone detector 880 can provide an indication (e.g., sound, haptic, communication to the communication device 884 (see FIG. 8), or the like) of whether the kidney stone 106 is present. The indication can be provided to the patient, medical personnel, or other person. The patient can know when to visit a medical facility to have the kidney stone 106 broken up or otherwise removed.

While embodiments are described with reference to kidney stone managements, the embodiments are applicable to other applications. One such application is gall stone detection and management. Such stone management can include delivery of a stone blocker using a duodenoscope, cholangioscope, or the like. The stone blocker can be situated in the gall bladder, pancreas, or other organ.

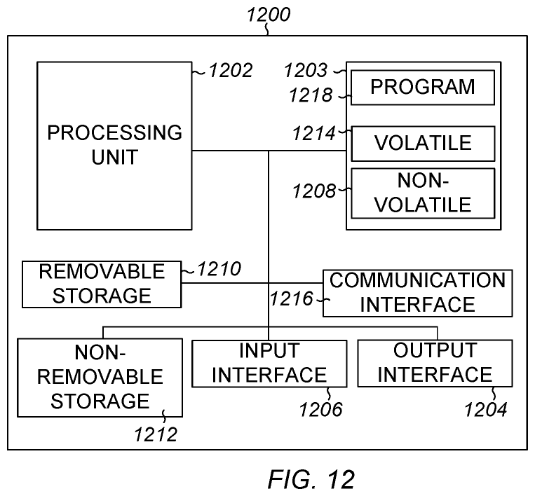

FIG. 12 illustrates, by way of example, a block diagram of an embodiment of a machine 1200 (e.g., a computer system) to implement one or more embodiments. The machine 1200 can implement a technique for stone 106 detection. The stone detector 880, 882 or the communication device 884 can include one or more components of the machine 1200.

One example machine 1200 (in the form of a computer), can include a processing unit 1202, memory 1203, removable storage 1210, and non-removable storage 1212. Although the example computing device is illustrated and described as machine 1200, the computing device can be in different forms in different embodiments. For example, the computing device can instead be a smartphone, a tablet, smartwatch, or other computing device including the same or similar elements as illustrated and described regarding FIG. 12. Devices such as smartphones, tablets, and smartwatches are generally collectively referred to as mobile devices. Further, although the various data storage elements are illustrated as part of the machine 1200, the storage can also or alternatively include cloud-based storage accessible via a network, such as the Internet.

Memory 1203 can include volatile memory 1214 and non-volatile memory 1208. The machine 1200 can include—or have access to a computing environment that includes—a variety of computer-readable media, such as volatile memory 1214 and non-volatile memory 1208, removable storage 1210 and non-removable storage 1212. Computer storage includes random access memory (RAM), read only memory (ROM), erasable programmable read-only memory (EPROM) & electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices capable of storing computer-readable instructions for execution to perform functions described herein.

The machine 1200 can include or have access to a computing environment that includes input 1206, output 1204, and a communication connection 1216. Output 1204 can include a display device, such as a touchscreen, that also can serve as an input device. The input 1206 can include one or more of a touch screen, touchpad, mouse, keyboard, camera, one or more device-specific buttons, one or more sensors integrated within or coupled via wired or wireless data connections to the machine 1200, and other input devices. The computer can operate in a networked environment using a communication connection to connect to one or more remote computers, such as database servers, including cloud-based servers and storage. The remote computer can include a personal computer (PC), server, router, network PC, a peer device or other common network node, or the like. The communication connection can include a Local Area Network (LAN), a Wide Area Network (WAN), cellular, Institute of Electrical and Electronics Engineers (IEEE) 802.11 (Wi-Fi), Bluetooth, or other networks.

Computer-readable instructions stored on a computer-readable storage device are executable by the processing unit 1202 (sometimes called processing circuitry) of the machine 1200. A hard drive, CD-ROM, and RAM are some examples of articles including a non-transitory computer-readable medium such as a storage device. For example, a computer program 1218 can be used to cause processing unit 1202 to perform one or more methods or algorithms described herein.

### ADDITIONAL NOTES AND EXAMPLES

Example 1 includes a medical apparatus comprising a kidney stone blocker configured to (i) remain in a human kidney after deployment and (ii) prevent passage of a kidney stone greater than a predetermined size into a ureter while allowing urine to pass to the ureter, and an elongated, tubular member mechanically coupled to the kidney stone blocker and configured to be situated in the ureter.

In Example 2, Example 1 can further include, wherein the kidney stone blocker includes a mesh screen.

In Example 3, Example 2 can further include, wherein the kidney stone blocker includes multiple mesh screens, at least two of the mesh screens configured to block kidney stones of respective, different minimum sizes.

In Example 4, Example 3 can further include, wherein the multiple mesh screens are arranged such that a first mesh screen of the multiple mesh screens is situated further from the ureter than a second mesh screen of the multiple mesh screens, the second mesh screen configured to prevent passage of smaller kidney stones than the first mesh screen.

In Example 5, at least one of Examples 1-4 can further include an anchor mechanically coupled to the member, the anchor configured to be retained in a bladder.

In Example 6, at least one of Examples 1-5 can further include, wherein the member is at least one of longitudinally extendable or elastic.

In Example 7, at least one of Examples 1-6 can further include, wherein the kidney stone blocker includes at least one of a barb, hook, tine, or stem configured to engage with the kidney and retain the kidney stone blocker in the kidney.

In Example 8, at least one of Examples 1-7 can further include, wherein the kidney stone blocker is expandable so as to fit through the ureter in a non-expanded form and to be retained in the kidney in an expanded form.

In Example 9, Example 8 can further include, wherein the kidney stone blocker includes a hydrophilic coating.

In Example 10, at least one of Examples 1-9 can further include a stone breaking device situated in the member, the stone breaking device configured to break the kidney stone into a plurality of smaller kidney stones.

In Example 11, Example 10 can further include, wherein the stone breaking device includes at least one of an optical device, an audio device, or a mechanical device.

Example 12 includes a method comprising providing a stone blocker into a ureter, the stone blocker formed with at least one opening for preventing passage of a kidney stone larger than the openings into the ureter while allowing urine to pass to the ureter, positioning the stone blocker within a kidney, and maintaining the stone blocker within the kidney to retain, in the kidney, the kidney stone larger than the at least one opening.

In Example 13, Example 12 can further include expanding, by activating an expansion mechanism, the stone blocker after positioning the stone blocker within the kidney.

In Example 14, Example 13 can further include, wherein the expansion mechanism includes at least one of a balloon or a spring-loaded wire.

In Example 15, at least one of Examples 12-14 can further include detecting whether the kidney stone is present, and responsive to detecting the kidney stone is present, breaking up the kidney stone into smaller kidney stones.

In Example 16, at least one of Examples 12-15 can further include detecting whether the kidney stone is present, and responsive to detecting the kidney stone is present, transmitting an indication to a communication device that the kidney stone is present.

Example 17 can include a system comprising a kidney stone detection device configured to determine whether a kidney stone is present in a kidney of a patient, and a kidney stone blocker configured to (i) remain in a kidney after deployment and (ii) prevent passage of the kidney stone into a ureter while allowing urine to pass to the ureter.

In Example 18, Example 17 can further include, wherein the kidney stone detection device produces at least one of sound waves or light to determine whether a kidney stone is present in a kidney of a patient.

In Example 19, at least one of Examples 17-18 can further include an elongated, tubular member mechanically coupled to the kidney stone blocker, the member sized to be situated in a ureter.

In Example 20, at least one of Examples 17-19 can further include, wherein the kidney stone blocker is expandable so as to fit through the ureter in a non-expanded form and to be retained in the kidney in an expanded form.

The preceding description and the drawings sufficiently illustrate specific embodiments to enable those skilled in the art to practice them. Other embodiments can incorporate structural, logical, electrical, process, and other changes. Portions and features of some embodiments can be included in, or substituted for, those of other embodiments. Embodiments set forth in the claims encompass all available equivalents of those claims.

Throughout this specification, plural instances can implement components, operations, or structures described as a single instance. Although individual operations of one or more methods are illustrated and described as separate operations, one or more of the individual operations can be performed concurrently, and nothing requires that the operations be performed in the order illustrated. Structures and functionality presented as separate components in example configurations can be implemented as a combined structure or component. Similarly, structures and functionality presented as a single component can be implemented as separate components. These and other variations, modifications, additions, and improvements fall within the scope of the subject matter herein.

Although an overview of the inventive subject matter has been described with reference to specific example embodiments, various modifications and changes can be made to these embodiments without departing from the broader scope of embodiments of the present disclosure. Such embodiments of the inventive subject matter can be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments can be used and derived therefrom, such that structural and logical substitutions and changes can be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” can be construed in either an inclusive or exclusive sense. Moreover, plural instances can be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and can fall within a scope of various embodiments of the present disclosure. In general, structures and functionality presented as separate resources in the example configurations can be implemented as a combined structure or resource. Similarly, structures and functionality presented as a single resource can be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain principles involved and their practical applications, to thereby enable others skilled in the art to best utilize the various example embodiments with various modifications as are suited to the particular use contemplated.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in response to detecting,” depending on the context. Similarly, the phrase “if it is determined” or “if [a stated condition or event] is detected” can be construed to mean “upon determining” or “in response to determining” or “upon detecting [the stated condition or event]” or “in response to detecting [the stated condition or event],” depending on the context.

What is claimed is:

1. A medical apparatus comprising:
a kidney stone blocker configured to (i) remain in a human kidney after deployment and (ii) prevent passage of a kidney stone greater than a predetermined size into a ureter while allowing urine to pass to the ureter; and
an elongated, tubular member mechanically coupled to the kidney stone blocker and configured to be situated in the ureter.

2. The medical apparatus of claim 1, wherein the kidney stone blocker includes a mesh screen.

3. The medical apparatus of claim 2, wherein the kidney stone blocker includes multiple mesh screens, at least two of the mesh screens configured to block kidney stones of respective, different minimum sizes.

4. The medical apparatus of claim 3, wherein the multiple mesh screens are arranged such that a first mesh screen of the multiple mesh screens is situated further from the ureter than a second mesh screen of the multiple mesh screens, the second mesh screen configured to prevent passage of smaller kidney stones than the first mesh screen.

5. The medical apparatus of claim 1, further comprising an anchor mechanically coupled to the member, the anchor configured to be retained in a bladder.

6. The medical apparatus of claim 1, wherein the member is at least one of longitudinally extendable or elastic.

7. The medical apparatus of claim 1, wherein the kidney stone blocker includes at least one of a barb, hook, tine, or stem configured to engage with the kidney and retain the kidney stone blocker in the kidney.

8. The medical apparatus of claim 1, wherein the kidney stone blocker is expandable so as to fit through the ureter in a non-expanded form and to be retained in the kidney in an expanded form.

9. The medical apparatus of claim 8, wherein the kidney stone blocker includes a hydrophilic coating.

10. The medical apparatus of claim 1, further comprising a stone breaking device situated in the member, the stone breaking device configured to break the kidney stone into a plurality of smaller kidney stones.

11. The medical apparatus of claim 10, wherein the stone breaking device includes at least one of an optical device, an audio device, or a mechanical device.

12. A method comprising:
providing a stone blocker into a ureter, the stone blocker formed with at least one opening for preventing passage of a kidney stone larger than the openings into the ureter while allowing urine to pass to the ureter;
positioning the stone blocker within a kidney; and
maintaining the stone blocker within the kidney to retain, in the kidney, the kidney stone larger than the at least one opening.

13. The method of claim 12, further comprising expanding, by activating an expansion mechanism, the stone blocker after positioning the stone blocker within the kidney.

14. The method of claim 13, wherein the expansion mechanism includes at least one of a balloon or a spring-loaded wire.

15. The method of claim 12, further comprising:
detecting whether the kidney stone is present; and
responsive to detecting the kidney stone is present, breaking up the kidney stone into smaller kidney stones.

16. The method of claim 12, further comprising:
detecting whether the kidney stone is present; and
responsive to detecting the kidney stone is present, transmitting an indication to a communication device that the kidney stone is present.

17. A system comprising:
a kidney stone detection device configured to determine whether a kidney stone is present in a kidney of a patient; and
a kidney stone blocker configured to (i) remain in a kidney after deployment and (ii) prevent passage of the kidney stone into a ureter while allowing urine to pass to the ureter.

18. The system of claim 17, wherein the kidney stone detection device produces at least one of sound waves or light to determine whether a kidney stone is present in a kidney of a patient.

19. The system of claim 17, further comprising an elongated, tubular member mechanically coupled to the kidney stone blocker, the member sized to be situated in a ureter.

20. The system of claim 17, wherein the kidney stone blocker is expandable so as to fit through the ureter in a non-expanded form and to be retained in the kidney in an expanded form.
